(19) United States
(12) Patent Application Publication
(10) Pub. No.: US 2021/0214291 A1
(43) Pub. Date: Jul. 15, 2021

(54) Rapid Thermal Isomerization of Lycopene

(71) Applicant: Michael Lloyd, Decatur, GA (US)

(72) Inventor: Michael Lloyd, Decatur, GA (US)

(21) Appl. No.: 16/752,139

(22) Filed: Jan. 24, 2020

Related U.S. Application Data
(60) Provisional application No. 62/959,391, filed on Jan. 10, 2020.

(51) Int. Cl. C07C 5/25; B01J 19/12

(52) U.S. Cl. CPC C07C 5/25; C07B 37/08; B01J 19/126

(57) ABSTRACT

The use of lycopene has been demonstrated to be effective in decreasing risk factors associated with cardiovascular disease, skin cancer and prostate cancer in mammals. Lycopene is difficult to solubilize in its native trans-lycopene form. Cis-lycopene, formed by applying thermal energy generated by excitation of polar molecules through microwave-assisted processing, appears in several isomeric forms. The cis isomers are effective in improving lycopene micellularization, bioaccessibility and mammalian absorption. The cis isomers are effective in improving vascular circulation of lycopene by way transport vesicle low density lipo-protein (LDL). Lycopene-based ingredients, end products, functional foods, medical foods and nutraceuticals, containing isomerized cis-lycopene can be used in place of ingredients with more naturally abundant trans-lycopene as phytonutrient, micronutrient and antioxidant delivery vehicles through dietary consumption to improve the outcomes of a variety of conditions, including hypertension, cardiovascular disease, skin cancer, prostate cancer, macular degeneration and related proinflammatory conditions.

### PARENT CASE

This patent application claims priority of Provisional Patent Application 62/959,391 filed Jan. 10, 2020.

Full benefit of priority from said parent provisional application is sought herein.

The present invention relates to a process for the rapid thermal isomerization of a mixture of tomato homogenate all-trans-lycopene and its cis-isomers of any composition to increase the proportion of cis or Z isomers, wherein the isomerization takes place in a polar solvent in which lycopene is insoluble or only slightly soluble.

### DESCRIPTION

### Field of Invention

The present invention relates to a process for microwave-assisted thermal cis-isomerization of a mixture of red tomato homogenate containing abundant all-trans-lycopene and its lesser abundant cis-isomer of any composition. Lycopene is a carotenoid which occurs naturally inter alia in red tomatoes.

There is to date no method by which lycopene containing fruit, vegetable or red flesh tomatoes containing majority trans-lycopene in its crystal form can be rapidly converted efficiently into the cis-lycopene isomers and increasing cis-isomer ratio compared to trans-lycopene using thermal energy transfer leading to increased mammalian absorption potential.

### Background of Invention

Carotenoids have important biological functions, with roles in light capture, antioxidative activity and protection against free radicals, synthesis of plant hormones and as structural components of the membranes. They are lipid soluble compounds that play an important role in human health and nutrition. Plants and algae utilize carotenoids to absorb light energy used in photosynthesis and protect chlorophyll form photo damage (Armstrong, 1996). In mammals, carotenoids are obtained from the diet and are stored in fatty tissues. As it relates to the human diet, carotenoid absorption is improved when consumed with fat in a meal (Jayarajan, 1980).

Improving the absorption and vascular circulation of carotenoid antioxidants could aid in improving the outcomes of individuals with CVD risk factors and proinflammatory conditions associated with aging. Cardiovascular disease is often associated with the diagnosis of metabolic related disorders such as diabetes and obesity. Cardiovascular disease (CVD) is the #1 cause of death in America. Nearly one in three US. adults, or 68 million persons, have hypertension; and nearly 30% of all US adults are pre-hypertensive (CDC, Vital signs hypertension, 2011; Roger, 2012). Hypertension is a major contributor to cardiovascular diseases, which are a leading cause of death, disability, and health-care costs in the United States (Heidenreich, 2011). Epidemiological evidence demonstrates that greater intake of carotenoids offers protection against cardiovascular disease, cancer, macular degeneration and neurodegenerative diseases (Krinsky, 2005).

Lycopene derived from tomatoes is the most commonly consumed carotenoid in the US. Lycopene exhibits the highest antioxidative properties among all dietary carotenoids (Miller, 1996; Bohm, 2002). Lycopene activates important mechanisms in the human body that protect against pathogenesis of degenerative diseases, especially coronary heart diseases, cancers and an array of other free radical-mediated conditions (Halliwell, 1997) (Kritchevsky, 1999). In vivo studies report that tomato products improved the antioxidative activity of the plasma and reduced LDL oxidation in healthy men (Lee, 2000; Bub, 2000). Tomato value-added products such as ketchup, bbq sauce, salsa, pizza sauce represent the majority of carotenoid-based foods typically consumed by Americans. Tomato farmers in California grow roughly 300,000 acres for tomato processors producing 12 million tons (23 billion lbs.) of product annually, worth an estimated $8 billion (Hartz, 2008; Carter, 2008). California accounts for over 90% of U.S. production and 35% of world production (Hartz, 2008). Tomato processing is a high value market in the US, yet the market's full potential related to carotenoid heart health benefits is not represented in many commonly consumed processed food products.

Tomatoes possess many beneficial phytonutrients but the most well studied is lycopene. Lycopene exists predominately in either the trans or cis isoforms. Trans-lycopene isoform is abundant in the native unprocessed tomato. As tomatoes are processed and/or cooked, the trans-lycopene is converted to the more bioavailable cis-lycopene isomer form, which has been shown to increase absorption by 2 to 3-fold. (Gartner, 1997; Stahl, 1992; Rao, 2002). Studies by Burri and Ishida (2009) sponsored by Agricultural Research Services showed the tangerine tomato's biofortified tetra-cis-lycopene is more efficiently absorbed by human bodies than is the trans-lycopene of red tomatoes (Burri, 2009).

Recently, studies in the Schwartz lab at Ohio State investigating the bioavailability of cis-lycopene containing tomato products showed 8.5 times increase in absorption in human studies and it was concluded that tomato-based food products could be manipulated by temperature processing to favor the formation of specific isomer patterns to improve lycopene bioavailability (Cooperstone 2015, Cooperstone 2016, Unlu, 2007). Collectively, the findings in these studies allude to the significance of establishing a proficient heat induced cis-lycopene conversion bioprocessing system for the readily available and less expensive red tomatoes in the production of post-harvest sterile value-added tomato ingredients and end products.

Lycopene is the most potent antioxidant of the carotenoid family. It contains 11 conjugated double bonds arranged linearly in the center portion of the molecule and 2 unconjugated double bonds at each end (Nguyen, 1999). This highly unsaturated hydrocarbon carotenoid has a molecular formula of C₄₀H₅₆. Lycopene is the longest carotenoid in the family, and its configuration provides a singlet-oxygen free radical quenching ability twice as high as that of β-carotene and 10 times higher than that of α-tocopherol (Nguyen, 1999; Weisburger, 2002). The radical scavenging activities of lycopene are related to its ability to trap peroxyl radicals (ROO⁻) and diffuse oxygen radicals (O₂⁻) (Foot, 1968; Burton, 1984). Lycopene has been reported to deactivate an array of free radicals, such as hydrogen peroxide, nitrogen dioxide, thyl, and sulphonyl (Bohm, 1995; Lu, 1995; Mortenson, 1997). Additionally, multiple investigations demonstrate that lycopene is a more potent ROS scavenger than many other dietary carotenoids and other antioxidants (Di Mascio, 1989; Di Mascio, 1991; Devasagayam, 1992; Miller, 1996).

The per capita consumption of tomatoes in the US is 73 lbs and the major sources of lycopene in the typical American diet come from tomato products such as spaghetti sauce, tomato juice, pizza sauce, and ketchup. These foods provide over 80 percent of the lycopene consumed in the U.S. The half-life of lycopene is 7 to 14 days in human blood, and a diet absent of lycopene for 1 week significantly lowers plasma concentrations (Allen, 2002; Hadley, 2003; Rao, 2002; Schwedhelm, 2003). Interestingly, investigations by Hadley and colleagues (2003) suggest that 1 week of consuming processed tomato products low in lycopene resulted in increased levels of the circulating cis-lycopene concentration. Research investigating low dose lycopene intake by Rao and Shen (2002) report that lycopene intake of 5-20 mg produced a significant increase in serum lycopene levels for both ketchup and lycopene capsules. Moreover, these studies suggested lycopene mediates a protective effect by significantly reducing lipid and protein oxidations in the body (Rao, 2002).

Compared to trans-lycopene, the absorption potential of cis-lycopene is enhanced due to the lower tendency to self-aggregate and form crystals (Britton, 1995). The solubility of cis-isomers is high in the lipophilic phase; therefore, they are more efficiently incorporated into bile acid micelles, pass across the intestinal barrier via passive diffusion, and are preferentially incorporated into chylomicrons (Bohm, 1999; Boileau, 2002, Cartner, 1997). Lycopene, as well as other carotenoids are absorbed through enterocyte uptake as well (Johnson, 1997; Tyssandier, 2002).

After gaining access to the body via lymphatic system or portal circulation, carotenoids accumulate in the liver, were they are packaged and released into circulation with lipoprotein particles. In blood plasma, lycopene (and other carotenoids) are mainly associated with low density lipoproteins (LDL) and very low-density lipoproteins (VLDL). The lycopene-lipoprotein interaction truly represents a symbiotic relationship. While the lipoproteins provide lycopene a transport mechanism to specific tissues, the lycopene protects the lipoprotein from oxidative damage (Agarwal, 2001). Carotenoids are transported to various tissue sites that have many low-density lipoprotein receptors and/or a high rate of lipoprotein uptake such as adrenal, prostate, lung, kidney, pancreas, and ovary (Gerster, 1997; Schmitz, 1993; Erdman, 1993). Lycopene is deposited in the liver, lungs, prostate gland, colon, and skin in the human body, and its concentration in body tissues tends to be higher than those of all other carotenoids (Rao, 1998; Rao, 2002; Shi, 2002; Shi, 2003). The protective role of lycopene to LDL in circulation is critical to decreasing the risk of coronary heart disease. Oxidized LDLs are directly involved in the formation of foam cells and arterial plaques, which increase the risk of cardiovascular disease (halal, 1996; Parthasarathy, 1998; Ames, 1995). Oxidation of LDL is responsible for the increase presence of activated macrophages and is believed to contribute to the development of atherosclerosis (Santanam, 2002). This is plausible because macrophage receptors preferably recognize the oxidized LDL form and could, therefore, be taken up by vascular tissue macrophages to induce the fatty-streak lesion of atherosclerosis (Lavy, 1993). The development of atherosclerotic plaques along arterial walls often leads to a decrease in vascular lumen size, which can result in elevated blood pressure and cardiovascular disease.

Studies suggest lycopene protects the body from lipid peroxidation (Bhuvaneswari, 2001; Velmurugan, 2002; Yeh, 2002). This is significant because several lipid peroxidation products were identified as cytotoxic and genotoxic and were shown to play an important role in the etiology of several chronic diseases, including acute coronary syndromes (Tsimikas, 2003; Boyd, 1991). Lipid peroxidation refers to the oxidative degradation of lipids. During this process free radicals steal electrons from the lipids in cell membranes, resulting in cell damage. Fortunately, lipophilic antioxidants can provide protection as they are embedded entirely within the nonpolar inner environment of the membrane and exhibit limited mobility. In the case of obese individuals, due to their excess stored energy, lipid cellular components would be likely in abundance and thus prone to oxidative damage; as chronic low-level inflammation is a hallmark of obesity. Due to the characteristic lipophilic properties of lycopene, it appears that the localization of carotenoids in the lipophilic component of the cell provides a greater resistance for lipid and lipid proteins to oxidative damage (Clevidence, 1993; Ribaya-Mercado, 1995). The availability and accessibility of bioavailable lycopene in systemic circulation throughout the vasculature may provide an additional layer of protection from oxidative damage.

Lycopene's lipid peroxidation protective effects may be accompanied by a lycopene induced activation of the cellular endogenous antioxidant defense system. This innate cellular defense mechanism that protects the body from oxidative damage consists of enzymes such as glutathione peroxidase, catalase, and superoxide dismutase. Velmurugan and colleagues (2002) conducted an animal study to examine lycopene antiperoxidation effect by using a potent carcinogen that produced toxic and highly diffusible reactive oxygen species. They reported a significant reduction in lipid peroxidation in both plasma and erythrocytes was observed in the animal group that was administrated with lycopene; and accompanied by enhanced levels of glutathione (GSH) and biotransformation enzymes, such as glutathione peroxidase (GPx), glutathione-S-transferase (GST), and glutathione reductase (GR) (Velmurugan, 2002). This is consistent with previous work by this group which shows the ingestion of lycopene significantly decreased the lipid peroxides and enhanced the activities of hepatic biotransformation enzymes in a hamster carcinogenesis model (Bhuvaneswari, 2001; Bhuvaneswari, 2002). With obesity being a chronically toxic condition that produces chronic medium to low doses of various inflammatory reactive oxygen species; the presence of circulating lycopene may induce similar pro-endogenous antioxidant defense system protective effects as observed in studies by Velmurugan et al (2002) and Bhuvaneswari et al (2002). In clinical studies, lycopene has been shown to activate super oxide dismutase (SOD) activity. In studies by Kim et al (2011), patients received a daily 6 mg or 15 mg lycopene supplementation for 8 wks; and results showed that SOD activity increased by 1.73 units per milliliter and 2.37 units per milliliter, respectively. The results were correlated with a significant improvement in endothelial function and a 57% reduction in C-reactive protein (CRP) levels in the high dose group (Kim, 2011). The beneficial effects of lycopene displayed in this study are significant and relevant to obesity and cardiovascular disease, as high CRP levels are positively correlated with obesity and the progression of atherosclerosis (Visser, 1999; Cook, 2000; Rohde, 1999). Moreover, the ability of low to moderate levels of dietary lycopene to upregulate SOD activity further substantiates the suggested protective effect dietary lycopene may provide beyond its traditional free radical quenching activities.

### Thermal Processing of Tomato Products

Recent advances in thermal processing technology have resulted in the establishment of methods possessing the potential to rival standard bioprocessing techniques used in today's food industry (Richardson, 2001). A significant factor in producing a more nutrient rich tomato-based condiment is selecting the way the product is processed post formulation. Tomatoes are the key ingredient, therefore a processing technique that facilitates the conversion and protection of lycopene, is paramount. Within the pericarp tissue of the ripe tomato fruit, lycopene is localized in the chloroplasts, in the form of fine crystals, which are associated with the membrane structure (Bouvier, 1998). While in the crystalline formation, lycopene is configured in its most thermodynamically stable trans-lycopene form. This structurally “grounded state” accounts for 90-96% of total lycopene in red tomato fruit, and is a predominant factor influencing the accessibility of lycopene to the human body. During mechanical and thermal processing, it is hypothesized stable lycopene isoform is freed from the fleshy membrane-bound matrix and isomerized to a more energy-rich state known as cis-lycopene isomers. Under the influence of heat, light, or certain chemical reactions, seven bonds comprised within the natural trans-lycopene molecule can perhaps be isomerized to the mono- or poly-cis form. This is significant because cis isomers of lycopene have physical characteristics and chemical behaviors distinct from the all-trans structure, and in human serum and tissue cis-lycopene isomers contribute more than 50% of total lycopene (Shi, 2000; Schierle, 1996). Data from research studies show that the human body better absorbs cis isomers of lycopene than all-trans form (Boileau, 2002; Cooperstone, 2015).

During the processing of tomatoes to value added products, heat intensity exposure and time duration are the most critical factors influencing isomerization or degradation. A study by Shi et al (2003) involving dissolved extracted lycopene in canola oil and heated to 25° C., 100° C., or 180° C. produced results that suggest degradation of lycopene was the main mechanism of lycopene loss when heated above 100° C. Conversely, at lower heating temperature of 70° C., Schierle et al (1996) reported data that shows cis isomerization levels increased proportionally to heating time. The presence of certain macromolecules in tomatoes may provide additional protection for lycopene during heat treatment. It was observed that lycopene loss was less while heating tomato pulp in comparison to heating lycopene in organic solution (Cole, 1957; Cole, 1957). In a separate study by Shi et al (2002), tomato puree was subjected to heat treatments of 90° C., 110° C., 120° C. and 150° C. for 1-6 hours to characterize isomerization and degradation of lycopene. The results indicate that the concentration of total lycopene steadily decreases with treatment (the higher the temperature, the faster the degradation), while the cis isomer levels increased but only during the first 2 hours of heating. The authors suggested that oxidation of lycopene was the main mechanism of lycopene loss when heated above 100° C., and that an optimum heating condition could be found to promote cis isomerization in tomato-based foods (Shi, 2002; Shi, 2003). Data from Schwartz's laboratory indicates that the trans-lycopene isomer is relative stable to isomerization at temperatures between 50 and 100° C. (Nguyen, 1999). Conversely, studies from this same lab reported thermal processing influenced isomerization of lutein and beta-carotene, but not lycopene (Nguyen, 2001).

### Microwave Thermal Processing

Microwave assisted thermal processing offers similar benefits to conventional methods, but with improved product quality and reduced time of exposure to energy (Canumir, 2002). Several studies have successfully been conducted on the microwave pasteurization of fruit juices as it preserves the natural organoleptic characteristics of the juice and reduces the time of exposure to energy, with the subsequently lower risk of losing essential thermolabile nutrients (Igual, 2010). It is generally accepted with microwave-assisted heating the come-up time for sterilization temperature range is shorter comparted to conventional heating, thus limiting heat exposure time. This occurs because the product is being heated from the inside out using polar and charged molecules within the fruit puree matrix. Microwaves are electromagnetic radiation waves with frequencies that lie between infrared and radio and TV waves. The principle of application of microwave radiation to fruit puree sterilization is that water present in food materials act as an electric dipole, which contains both positively and negatively charged molecules. When an electromagnetic radiation is passed through the food, heat energy is produced due to intermolecular frictions resulting from the movement of electrical charges produced by forces of attraction and repulson (Datta, 2013). Another mechanism of heating is due to ionic conduction. The application of electromagnetic field causes migration of the ions towards oppositely charged regions. This results in release of heat due to multiple billiard ball-like collisions and disruption of the H-bonds in water (Venkatesh, 2004). The amount of heat produced in food during microwave heating is proportional to food matrix's dielectric properties, including dielectric constant and dielectric loss factor (Tang, 2005).

Studies performed by Kumnar et al (2008) showed that thermophysical and dielectric properties of tomato value added food materials and ingredients are suitable for microwave heating at sterilization temperatures. Unlike conventional thermal sterilization, microwave heating causes uniform heating of the entire volume of the food, and provided water content is adequate, the energy is absorbed very fast, which causes rapid heating (Venkatesh, 2004). Volumetric heating is the term that has been given to this type of efficient energy transfer within food processing, as it allows internal heating instead of transferring heat from a secondary medium (Ramaswamy, 2014). The ability of microwave or volumetric heating to transfer energy uniformly and rapidly allows for an opportunity to identify ideal conditions for standardized lycopene isomerization conditions that stop short of oxidizing or degrading bioactive antioxidants and desirable organoleptic characteristics.

### SUMMARY OF THE INVENTION

The subject of invention relates to a method of thermal processing tomato or lycopene containing homogenate material in water or with water and oil using electromagnetic radiation and/or ionic conduction resulting in release of heat due to multiple billiard ball-like collisions and disruption of hydrogen bonds in a polar solvent (including but not limited to water), as observed with microwave-assisted heating or volumetric heating resulting in an increase in cis-isomerization conversion and improved mammalian absorption capacity of lycopene in red tomato or lycopene containing homogenate starting material post processing.

### DETAILED DESCRIPTION OF INVENTION

Theoretically lycopene can assume 211 or 2048 geometrical configurations, due to the 11 conjugated carbon-carbon double bonds in its backbone (Omani, 2005). Lycopene biosynthesis in plants leads to the all-trans-form that is independent of its thermodynamic stability. In human plasma, lycopene is an isomeric mixture, containing at least 60% of the total lycopene as cis-isomers (Kim, 2012).

All-trans, 5-cis, 9-cis, 13-cis, and 15-cis are the most commonly identified isomeric forms of lycopene with the stability sequence being 5-cis>all-trans>9-cis>13-cis>15-cis>7-cis>11-cis (Agarwal, 2000). The 5-cis-form is thermodynamically more stable than the all-trans-isomer, nevertheless a large number of geometrical isomers are theoretically possible for all-trans lycopene with limitations to cis-trans isomerization by steric hinderance of ethylenic groups of certain lycopene molecules (Agarwal, 2000).

In fruit such as tomatoes lycopene exists in chromoplast as ratio of E/Z or trans/cis isomers depending on cultivar and degree of ripeness. Our lab has observed homogenized untreated samples with a ratio of trans to cis at 96:4, respectively. Studies performed by Honda et al on isomerization of trans-lycopene, purified from tomato paste, was investigated with organic solvents and the isomerization ratios to the cis-isomer of lycopene ranged from 11.4% at 4 C to 77.8% at 50 C for 24 hrs (Honda, 2015).

Previous state of the art in lycopene isomerization process approved for patent protection was designed to convert existing Z or cis-lycopene isomers in lycopene containing material such as tomatoes to E or trans-lycopene isomers (U.S. Pat. No. 7,126,036). These processes involving organic solvents and traditional thermal processes entailed several ours of processing in the range of 2-160 hours, preferably 16 to 40 hours at select temperatures.

There is to date no method by which a mixture of trans-lycopene isomers or else individual trans-lycopene can be converted efficiently in minutes to cis-lycopene isomer forms. There is to date no method by which a mixture of cis-lycopene or else individual cis-lycopene can be excited efficiently in minutes into other cis-lycopene isomer forms.

It is an object of the present invention to develop an efficient method or isomerizing the trans-lycopene form into cis-lycopene form which does not have the described prior art disadvantages and makes it possible to use the more energy efficient and cost-effective thermal energy transfer isomerization process for lycopene.

We have found that this object is achieved by a process for the volumetric heating and/or microwave-assisted rapid thermal isomerization of a mixture of trans-lycopene and its cis-isomers of any composition to increase the proportion of cis-lycopene isomers, wherein the isomerization takes place in a polar solvent in which lycopene is insoluble or slightly soluble.

The invention thus relates to a process for the thermal isomerization of trans-lycopene and its cis-lycopene isomers of any composition to increase the proportion of cis-lycopene, wherein the isomerization takes place in a polar solvent.

The process of the invention makes use of a suspension of lycopene in a polar solvent in which lycopene is insoluble soluble or slightly soluble.

Polar solvents employed are water or water/oil.

Polar solvents are monounsaturated and polyunsaturated fatty acids such as edible oils including but not limited to olive oil, canola oil, sunflower oil, vegetable oil, safflower oil, grapeseed oil saffron oil, black cumin seed oil, cooking oils, omega 3 fatty acids, omega 6 fatty acids, lecithin, surfactants and fat soluble vitamins E, D, K, A.

Polar solvents employed can be alcohols such as C.sub.1 C.sub.8-alcohols, diols, polyols, amines, carbonates, sulfoxides or edible oils.

C.sub.1 C.sub.8-Alcohols are, for example, methanol, ethanol, ethylene glycol, glycerol, propanol, isopropanol, butanol, tert-butanol, pentanol, hexanol, heptanol, or octanol, and methanol, ethanol, or butanol are preferably employed. An example of a diol which can be employed is ethylene glycol. Polyols mean, for example, polyethylene glycol. Examples of amines are formamide, acetamide, methylformamide, methylacetamide, dimethylformamide, dimethylacetamide or y-butyrolactone. Carbonates mean, for example, ethylene carbonate or propylene carbonate. An example of a sulfoxide which can be used is dimethyl sulfoxide.

The process of the invention exploits the effect that microwave assisted heating excites polar molecules throughout the discontinuous phase of the homogenate composition which contains the continuous phase comprising the virtually insoluble trans-lycopene accompanied by 5-cis-lycopene in their respective most thermodynamically stable forms. Thermogenesis formed from polar and ionic molecules activated by microwave generation excites lycopene isomers to change conformation favoring increases in cis-isomer content and cis-isomerization. In this case it is possible, if the temperature is sufficiently high, for there to be selective isomerization of cis-lycopene isomers, because the trans-lycopene isomers bound in the crystal have a considerably higher isomerization activation energy. Owing to the properties of crystalline trans-lycopene, solubilization with oil or polyunsaturated triglycerides lowers trans-lycopene isomerization activation energy thus allowing the process of the invention to exploit the effect of microwave assisted heating or volumetric heating to effectively excite polar and ionic molecules throughout the continuous and discontinuous phases of the fruit or vegetable homogenate containing lycopene. Overall, the isomerization equilibrium can thus be shifted towards the cis-lycopene isomer.

The isomerization temperature is between 40 and 180 degree C., preferably between 60 and 120 degree C. The isomerization can be carried out both under atmospheric pressure and under elevated pressure, preferably under pressures of from 0 to 10 bar. The isomerization process duration is between 0.25 minutes and 2,880 minutes, preferably between 2 minutes and 1,440 minutes.

A suspension of lycopene in a polar solvent in which lycopene is insoluble or only slightly soluble is prepared after shear stress homogenization processing of fruit or vegetable material to give lycopene directly in this polar solvent. Hydrophobic compounds, oil and/or amphiporus surfactant processing aids can be added during the homogenization process within a range between 0.25%-75% of lycopene, preferably between 1%-60% of lycopene to improve lycopene solubilization in polar solvent.

It is then possible for lycopene, in various ratios of amounts in relation to the polar solvent, preferable as 1 to 98% strength suspension of lycopene in the polar solvent, to be isomerized by microwave or dielectric property influenced volumetric heating or thermogenesis.

This can then be followed by 8 different variants: firstly, the homogenate suspension can be isomerized directly by microwave or volumetric heating. An alternative is to add edible oil or polyunsaturated fatty acids and subsequently isomerize by heating. In the third variant, the surfactant, for example, lecithin is added to the homogenate suspension with or without oil, and then isomerization is carried out. The fourth variant, adding an alcohol, for example methanol, and subsequently to isomerize by heating. The fifth variant, the solvent incorporates, for example, n-butanol, and then is isomerization is carried out. The sixth variant, the solvent incorporates, for example, hexane then the isomerization is carried out. The seventh variant, the solvent incorporates, for example ethyl acetate then the isomerization is carried out. The eighth variant, the solvent incorporates, for example alkyl halides such as dichloromethane or chloroform then the isomerization is carried out.

The lycopene quality and quantity can be evaluated by cooling the suspension, and performing a lycopene extraction and washing, then determining the qualitative and quantitative content by HPLC measurement.

It was possible to increase 9-cis-lycopene isomer by 1010% in HR (Heated Roma) compared to control (unheated) (FIG. 3), increase 7-cis-lycopene isomer by 344% in HR compared to control (FIG. 2), and increase di-cis-lycopene isomer by 99.4% in HR compared to control (FIG. 6). Results from in-vitro digestibility assay confirmed increases in cis-lycopene isomerization translated into a 25% increase in relative bioaccessbility of cis-lycopene (FIG. 12) in HR. No significant increase in absolute bioaccessbility for total cis-Lycopene or trans-Lycopene was observed in HR compared to control (FIGS. 10 & 11).

It was possible to increase 9-cis-lycopene isomer by 758% in HRO (Heated Roma Oil) compared to control (unheated) (FIG. 3), increase 9′-cis-lycopene isomer by 50% in HRO compared to control (FIG. 4), increase 13-cis-Lycopene isomer by 40000% in HRO compared to control (FIG. 5), and increase di-cis-lycopene isomer by 182% in HRO compared to control (FIG. 6). Data shows a 350% increase of c-Lycopene-1 in HRO compared to control (FIG. 7). Results from in-vitro digestibility assay confirmed increases in cis-lycopene isomerization translated into a 100% increase in relative bioaccessbility of cis-lycopene in HRO (FIG. 12) and a 315% increase in total-Lycopene relative bioaccessbility in HRO compared to control (FIG. 12). Additionally, HRO treatment expressed a 200% increase in total cis-Lycopene isomer absolute bioaccessbility (FIG. 10) and a 220% increase in trans-Lycopene isomer absolute bioaccessbility compared to control (FIG. 11).

It was possible to increase 9-cis-lycopene isomer by 333% in HROL (Heated Roma Oil/Lecithin) compared to control (unheated) (FIG. 3), no significant increase 9′-cis-lycopene isomer in HROL compared to control (FIG. 4), increase di-cis-lycopene isomer by 81.9% in HROL compared to control (FIG. 6). Data shows a 270% increase of c-Lycopene-1 in HROL compared to control (FIG. 7). Results from in-vitro digestibility assay confirmed increases in cis-lycopene isomerization translated into a 362.8% increase in relative bioaccessbility of cis-lycopene in HROL (FIG. 12) and a 905.6% increase in total-Lycopene relative bioaccessbility in HROL compared to control (FIG. 12). Additionally, HROL treatment expressed a 650% increase in total cis-Lycopene isomer absolute bioaccessbility (FIG. 10) and 850% increase in trans-Lycopene isomer absolute bioaccessbility compared to control (FIG. 11).

It was possible to influence the cis/trans isomer ratio based upon processing conditions concerning the current invention. Analysis of isomer ratio in mg/100 g showed a cis/trans ratio of 1:15 for control, 1:12 for HR, 1:7 for HRO and 1:9 for HROL, which collectively represents lycopene crystalline structure solubilization and isomerization efficiency as cis/trans ratio is shifted in the direction of cis-lycopene at the expense of trans isomer. Moreover, it was possible identify a shift in cis/trans lycopene percentage ratio shift from 4%:96% in control, to 8%:92% in HR, 12%:88% in HRO and 10%:90% in HROL. This data is consistent with both increases in cis-isomer generation by microwave or volumetric mediated heating and increases observed in absolute and relative bioaccessbility of lycopene and its isomers in heat treated samples (HR, HRO, HROL) versus control untreated samples.

Cis-Lycopene isomerization is obtained by homogenizing lycopene containing fruit or vegetable and heating using microwave or volumetric mediated thermogenesis. Lycopene is a naturally occurring carotenoid, and is otherwise available to those of skill in the art. It may be obtained from a large variety of natural sources, including tomato.

The advantages which can thus be achieved overall by the volumetric heating and/or microwave-assisted thermal lycopene isomerization method of the invention are as follows: increase in more bioavailable yield of cis-lycopene isomer in processed lycopene containing material, decrease in isomerization activation energy requirement, decrease in thermal processing times necessary to achieve desired lycopene isomerization, decrease in cost of production due to rapid, efficient and identifiable processing end points defined by quantification of lycopene isomer quality, and increase in lycopene bioaccessiblility due to increase in micellularization efficiency of lycopene and its isomers resulting from the novel process invention.

In vitro bioaccessibility assay was used to simulate human oral, gastric and small intestinal digestion based on the method described by Hedren et al and Garrett et al. Tomatoes were subjected to a three-stage static in vitro digestion model, and lycopene was analyzed by HPLC-MS. The lycopene bioaccessibility of a sample is reported as a ratio (%) of the in vitro bioaccessible lycopene content to the corresponding lycopene content of the sample. This in vitro digestion model has been widely applied to fat-soluble micronutrients vitamin D, Vitamin E, vitamin K, fat-soluble carotenoids to model gastric and small intestinal release as well as micellarization (Reboul 2011, Failla 2008). In the small intestine, bile is required to incorporate lycopene into mixed micelles (Schachter 1964). Lycopene that has been released from the food matrix and solubilized in mixed micelles is available for subsequent absorption at the epithelial surface in the duodenum and jejunum (bioaccessible). While in vitro methodologies may not fully portray in vivo bioavailability, this approach offers a method to comparatively assess lycopene containing food matrixes with additional insight into confirming the impact processing including but not limited to rapid microwave-assisted or dielectric property mediated volumetric heat induced lycopene isomerization has on improving lycopene solubilization and micellularization leading to enhanced absorption capacity for lycopene antioxidants.

This invention has been disclosed in terms of specific embodiments, and generic description. The specific embodiments are not intended as limiting, and variations will occur to those of ordinary skill in the art without the exercise of inventive skill. Such variations remain within the scope of the invention, save as excluded by the recitations of the claims set forth. In particular, variations in isomerization procedure and specific condition will occur to those of ordinary skill in the art, without departing from the scope and spirit of the invention.

### REFERENCE CITED

### U.S. Patent Documents

U.S. Pat. No. 284,957 August 1958 Otto et al.
U.S. Pat. No. 5,166,445 November 1992 Meyer
U.S. Pat. No. 6,187,959 February 2001 Wegner et al.

### Foreign Patent Documents

382 67 Aug. 1990 EP
895 997 Feb. 1999 EP
Polgar, A. et al., “Isomerization of .beta.-Carotene. Isolation of a Stereoisomer with Increased Adsorption Affinity”, J. Am. Chem. Soc., 1942, vol. 64, 1856-1861. cited by examiner.
Doering et al., “Thermal Interconversions among 15-cis, 13-cis and all-trans-.beta.-Carotene: Kinetics, Arrhenius Parameters, Thermochemistry, and Potential Relevance to Anticarcinogenicity of all-trans-.beta.-Carotene”, J. Am. Chem. Soc., 1995, vol. 117, 2747-2757. cited by examiner.
Muller et al., “(E/Z)-Isomeric carotenes”, Pure & Appl. Chem., 1997, vol. 69, No. 10, 2039-2046. cited by examiner.

### Other References

Agrawal, S. & Rao, V. (2000): Tomato lycopene and its role in human health and chronic diseases. Canadian Medical Association Journal, 163: 739-744.
Agarwal A, Shen H, Agarwal S, Rao A V. Lycopene Content of Tomato Products: Its Stability, bioavailabillity and In Vivo Antioxidant Properties. Journal of Medicinal Food. 2001; vol 4(1):9-15.
Allen C M, Smith A M, Clinton S K, and Schwartz S J. Tomato consumption increases lycopene isomer concentration in breast milk and plasma of lactating women. J. Am. Diet. Assoc. 2002; 102:1257-1262.
Ames B N, Gold L S, Willet W C: Causes and prevention of cancer. Proc Natl Acad Sci USA. 1995; 92:5258-5265.
Annan, R. A., Webb, P., & Brown, R. (2014). Management of moderate acute malnutrition (MAM): current knowledge and practice. In CMAM Forum Technical Brief, 1-39.
Anthon G E, Barrett D M. (2010). Changes in pectin methylesterification and accumulation of methanol during production of diced tomatoes. Journal of Food Engineering, 97(3), 367-372.
Armstrong G A, Hearst J E (1996). Carotenoids 2: Genetics and molecular biology of carotenoid pigment biosynthesis. FASEB J. 10 (2):228-37
Arroyave, G. Mejia, L A. (2010). Five decades of vitamin A studies in the region of Central America and Panama. Food Nutrition Bulletin. 31(1), 118-29
Bengtsson A, Alminger M L, Svanberg U. (2009). In Vitro Bioaccessibility of B-Carotene from Heat-Processed Orange-Fleshed Sweet Potato. J. Agric. Food Chem. 2009, 57, 9693-9698
Bhuvaneswari V, Velmurugan B, Balasenthil S, Ramachandran C R, Nagini S. 2001. Chemopreventive efficacy of lycopene on 7,12-dimethylbenz[a]anthracene-induced hamster buccal pouch carcinogenesis. Fitoterapia, 72:865-874.
Bhuvaneswari V, Velmurugan B, Nagini S. 2002. Induction of glutathione-dependent hepatic biotransformation enzymes by lycopene in the hamster cheek pouch carcinogenesis model. J. Biochem. Mol. Biol. Biophys., 6:257-260.
Bohm V. and Bitsch R. 1999. Intestional absorption of lycopene from different matrices and interactions to other carotenoids, the lipid status, and the antioxidant capacity of human plasma. Eur. J Nutr., 38: 118-125.
Bohm V, Puspitasari-Nienaber N L, Ferruzi M G, Schwartz S J (2002) J Agric Food Chem 50:221-226
Bohm F, Tinkler J H, and Truscott T G. 1995. Carotenoids protect against cell membrane damage by the nitrogen dioxide radical. Nat. Med., 1:98-99
Boileau, A. C.; Merchen, N. R.; Wasson, K.; Atkinson, C. A.; Erdman, J. W., Jr. cis-Lycopene is more bioavailable than translycopene in vitro and in vivo in lymph-cannulated ferrets. J. Nutr. 1999, 129, 1176-1181.
Boileau T W M, Boileau A C, Erdman J W Jr: Bioavailability of all-trans and cis-isomers of lycopene. Exp Biol Med 2002; 227: 914-919.
Bouvier, F., Backhaus, R. A., and Camara, B. 1998. Induction and control of chromoplast-specific carotenoid genes by oxidative stress. J. Biol. Chem., 214:3-17.
Boyd N F, McGuire V. 1991. The possible role of lipid peroxidation in breast cancer risk. Free Radic. Biol. Med., 10:185-190.
Britton G. 1995. Structure and properties of carotenoids in relation to function. FASEB J., 9: 1551-1558.
Brown, M. J.; Ferruzzi, M. G.; Nguyen, M. L.; Cooper, D. A.; Eldridge, A. L.; Schwartz, S. J.; White, W. S. Carotenoid bioavailability is higher from salads ingested with full-fat than with fat reduced salad dressings as measured with electrochemical detection. Am. J. Clin. Nutr. 2004, 80, 396-403.
Bub A, Watzl B, Abrahamse L, Delincee H, Adam S, Wever J, Muller H, Rechkemmer G (2000) J Nutr 130:2200-2206
Burri B and Wood M. 2009. http://ars.usda.gov/is/AR/archive/feb11/lycopene0211.pdf
Burri, Betty Jane; Chapman, Mary H.; Neidlinger, Terry R.; Seo, Jung S.; Ishida, Betty K. International Journal of Food Sciences & Nutrition. May 2009 Supplement 1, Vol. 60, p 1-16.
Burton G W and Ingold, K U. 1984. Beta-Carotene: An unusual type of lipid antioxidant. Science, 224:569-573.
Canumir J A, Celis J E, de Bruijn J, Vidal L V. 2002) Pasteurisation of apple juice using microwaves. LWT-Food Science and Technology 35(5):389-392.
Carter C A, (2008) Economics of the California Processing Tomato Market. Giannini Foundation of Agricultural Economics, University of California).
Cartner C, Stahl W, Sies H. Lycopene is more bioavailable from tomato paste than from fresh tomatoes. Am J Clin Nutr 1997; 66: 116-22.
CDC Grand Rounds: Dietary Sodium Reduction-Time for Choice. Morbidity and Mortality Weekly Report. Feb. 10, 2012/vol. 61/No. 5.
CDC 2014. Summary Health Statistics for U.S. Adults: 2012. http://www.cdc.gov/nchs/data/series/sr_10/sr10_260.pdf
CDC. Vital signs: food categories contributing the most to sodium consumption-United States, 2007-2008. MMWR 2012; 61: 92-8.
CDC, Vital signs; prevalence, treatment, and control of hypertension-United States, 1999-2002 and 2005-2008. MMWR 2011; 60: 103-8.
Charm S E. (1971). The fundamentals of food engineering, Wesport: AVI Pub Co.
Clevidence B A, Bieri J G. 1993. Association of carotenoids with human plasma lipoproteins. Methods Enzymol., 214:33-46.
Clinton S K: Lycopene: Chemistry, biology, and implications for human health and disease. Nutr Rev 1998; 56:35-51.
Cole E R, Kapur N S: Stability of lycopene I, Degradation by oxygen. J Sci Food Agric 1957; 8:360-365.
Cole E R, Kapur N S: The stability of lycopene. II Oxidation during heating of tomato pulps. J Sci Food Agric 1957; 8:366-368.
Cook D G, Mendall M A, Whincup P H, Carey I M, Ballam L, Morris J E, et al. C-reactive protein concentration in children: relationship to adiposity and other cardiovascular risk factors. Atherosclerosis 2000; 149:139-50.
Cooperstone J I, Francis D M, Schwartz S J. Thermal processing differentially affects lycopene and other carotenoids in cis-lycopene containing, tangerine tomatoes. Food Chemistry 210 (2016) 466-472
Cooperstone J I, Ralston R A, Riedl K M, Haufe T C, Schweiggert R M, King S A, Timmers C D, Francis D M, Lesinsk G B, Clinton S K, Schwartz S J. Enhanced bioavailability of lycopene when consumed as cis-isomers from tangerine compared to red tomato juice, a randomized, cross-over clinical trial. Molecular Nutrition & Food Research 59, Number 4, (2015) 658-669, doi: 10.1002/mnfr.201400658
Cooperstone J L, Tolber K L, Riedl K M, Teegarden M D, Cichon M J; et al Tomatoes protect against development of UV-induced keratinocyte carcinoma via metabolic alterations. Scientific Reports (Nature Publisher Group); London Vol. 7, (July 2017): 1-9.
Coronel P, Simunovic J, Sandeep K P. 2003. Thermal profile of milk after heating in a continuous microwave unit. J Food Sci 68:1976-81
Coronel P, Truong V, Simunovic J, Sandeep K P, Cartwright G. 2005. Aseptic processing of sweet potato purees using a continuous flow microwave system. J Food Sci 70, Nr. 9:531-36
Craft, N., Dorey, C. K., 2004. Carotenoid, tocopherol, and retinal concentrations in elderly human brain. J. Nutr. Health Aging 8 (3), 156-162.
Diario Oficial, Quinta Sección, Secretaria de Agricultura, Ganaderia, Desarrollo Rural, Pesca y Alimentacion (SAGARPA), 31 de Diciembre de 2010.
Dary, O. (2002). Lessons learned with iron fortification in Central America. Nutrition Reviews, 60(7), (II) S30-S33
Davis E M, Croteau R. (2000). Cyclization enzymes in the biosynthesis of monoterpenes,
sesquiterpenes, and diterpenes. Topics in Current Chemistry. 209 pp. 53-95.
Datta A K, Rakesh V, (2013) Principles of microwave combination heating. Comprehensive Reviews in Food Science and Food Safety 12(1):24-39.
Devasagayam, T P, Werner T, Ippendorf H, Martin H D, and Sies H. 1992. Synthetic carotenoids,
novel polyene polyketones and new capsorubin isomers as efficient quenchers of singlet molecular oxygen. Photochem. Photobiol., 55:511-514.
Dhuique-Mayer C, Servent A, Messan C, Achir N, Dornier M, Mendoza Y. (2018). Bioaccessibility of Biofortified Sweet Potato Carotenoids in Baby Food: Impact of Manufacturing Process. Frontiers in Nutrition. October 2018. Vol 5:98
Di Mascio P, Kaiser S, and Sies H. 1989. Lycopene as the most efficient biological carotenoid singlet oxygen quencher. Arch. Biochem. Biophys., 274:532-538.
Di Mascio P, Murphy M E, and Sies, H. 1991. Antioxidant defense systems: The role of carotenoids, tocopherols, and thiols. Am. J. Clin. Nutr., 53:194S-200S.
Dimitrov N V, Meyer C, Ulirey D E, Chenoweth W, Michelakis A, Malone W, Boone C, Fink G (1988). Bioavailability of B-Carotene in humans. Am. J. Clin. Nutr. 48:298-304.
Ekesa B, Poulaert M, Davey M W, Kimiywe J, Van den Bergh I, Blomme G, et al. ioaccessibility of provitamin A carotenoids in bananas (Musa spp.) and derived dishes in African countries. Food Chem. (2012) 133:1471-7. doi: 10.1016/j.foodchem.2012.02.036
Early E., Holcomb R B., Willoughby C., Brooks J., A Market Evaluation of Barbecue Sauces. Oklahoma State University-Food Technology Fact Sheet. Oklahoma Cooperative Extension Service, Division of Agricultural Sciences and Natural Resources. FPAC-137
Erdman J W Jr, Bierer T L, Gugger E T. 1993. Absorption and transport of carotenoids. Ann. NY Acad. Sci., 691:76-85.
Erdman, J. W., et al., 2015. Lutein and brain function. Food 4, 547-564.
Failla M L, Huo T, Thakkar S K. 2008. In vitro screening of relative bioaccessibility of carotenoids from foods, Asia Pac. J. Clin. Nutr., 2008, 17, 200-203
Fasina O O, Farkas B E, Fleming H P. 2003. Thermal and dielectric properties of sweetpotato puree. Int J Food Prop 6:461-72
Feart C, Letenneur L, Helmer C, Samieri C, Schalch W, Etheve S, Delcourt C, Dartigues J, Barberger-Gateau P. Plasma Carotenoids Are Inversely Associated With Dementia Risk in an Elderly French Cohort. J Gerontol A Biol Sci Med Sci, 2016, Vol 71, N 5, 683-688.
Feng, S. (2015). M S thesis. Current Research Advances in Micronutrient Fortification on Sweetpotato Flour and Development of an Affordable Nutrition Product. Retrieved from Theses and Dissertations. NC State University.
Feng, S. (2018). Doctoral Dissertation. Chemical and Nutritional Analysis of Products Derived from Sweet Potatoes. Retrieved from Theses and Dissertations. NC State University
Food and Agriculture Organization, FAO. (2001). Report of a Joint FAO/WHO/UNU Expert Consultation: Human Energy Requirements. Rome, 17-24 Oct. 2001. FAO Rome.
Foot C S and Denny R W. 1968. Chemistry of singlet oxygen. VII. Quenching by β-carotene. J. Am. Chem. Soc., 90:6233-6235.
Galloway, R., & Mcguire, J. (1994). Determinants of compliance with iron supplementation-supplies, side-effects, or psychology. Social Science & Medicine, 39(3), 381-390
Garrett D A, Failla M L, Sarama R J. 1999. Development of an in vitro digestion method to assess carotenoid bioavailability from meals. Journal of agricultural and food chemistry 47 (10): 4301-4309
Garrow J S, James W P T, Ralph A. (2000). Human Nutrition and Dietetics. 10^{th} ed. Churchill, Livingstone, London
Gartner C, Stahl W, Sies H: Lycopene is more bioavailable from tomato paste than from fresh tomatoes. Am j Clin Nutr 1997; 66:116-122.
Gerster H. The potential role of lycopene for human health. J Am Cell Nutr. 1997; 16(2): 109-26.
Giovannucci E: Tomatoes, tomato-based products, lycopene, and prostate cancer: Review of the epidemiologic literature. J Natl Cancer Inst 1999; 91:317-331
Giovannucci E, Asccherio A, Rimm E B, Stampfer M J, Colditz G A, Willett W C. 1995. Intake of carotenoids and retinol in relation of risk of prostate cancer. J. Natl. Cancer Inst. 87: 1767-1776.
Guan Z, Biswas T, Wu F. 2017, The US Tomato Industry: An Overview of Production and Trade. Food and Resource Economics Department, UF/IFAS Extension. Retrieved from http://edis.ifas.ufl.edu/pdffiles/FE/FE102700.pdf on July 2019.
Hadley C W, Clinton S K, and Schwartz S J. 2003. The consumption of processed tomato products enhances plasma lycopene concentrations in association with a reduced lipoprotein sensitivity to oxidative damage. J. Nutr., 133: 727-732.
Halliwell B. (1997). Antioxidants and human disease: A general introduction. Nutr. Rev. 55:44-52.
Hamer et al. (2017). North Carolina Agricultural Statistics 2017. Retrieved July 2019, from https://www.nass.usda.gov/Statistics_by_State/North_Carolina/Publications/Annual_Statistical_Bulletin/AgStat2017.pdf
Hanson C, Lyden E, Furtado J, Van Ormer M, White K, Overby N, Anderson-Berry A. Serum Lycopene Concentrations and Associations with Clinical Outcomes in a Cohort of Maternal-Infant Dyads. Nutrients 2018, 10, 204; p 1-11 dio:10..3390/nu10020204
Hartz T, Miyao G, Mickler J, Lestrange M, Stoddard S, Nunez J, Aegerter B. Processing Tomato Production in California. (2008) UC Vegetable Research & Information Center. Vegetable Production Series. University of California Division of Agriculture and Natural Resources. Publication 7228. http://anrcatalog.ucdavis.edu
Hedren E, Mulokozi G, Svanberg U. 2002. In vitro bioaccessibility of carotenes from green leafy vegetables cooked with sunflower oil or red palm oil. International Journal of Food Science and Nutrition, 53, 445-453
Heidenreich P A, Trogdon J G, Khavjou O A, et al. Forecasting the future of cardiovascular disease in the United States: a policy statement from the American Heart Association. Circulation 2011; 123: 933-44.
Hermann, W. and Obeid, R. (2011). The mandatory fortification of staple foods with folic acid—a controversy in Germany. Dtsch Arztebl Intl. 108 (15), 249-254. http://www.ncbi.nlm.nih.gov.prox.lib.ncsu.edu/pmc/articles/PMC3088169/. Accessed January, 2014
Honda M, Takahashi N, Kuwa T, Munenori T, Inoue Y, Kumagai T. Spectral characterization of Z-isomers of lycopene formed during heat treatment and solvent effects on the E/Z isomerization process; Food Chemistry 171(2015) 323-329.
Igual M, Garcia-Martinez E, Camacho M M, and Martinez-Navarrete N, 2010. Effect of thermal treatment and storage on the stability of organic acids and functional value of grapefruit juice. Food Chemistry 198(2):291-299
Institute of Medicine. Strategies to reduce sodium intake in the United States. Washington, D.C.: The National Academies Press. 2010.
International Vitamin A Consultative Group (IVACG), XXI Meeting (2003) Improving the Vitamin A Status of Populations. USAID. Rockville, Md., USA;
International Vitamin A Consultative Group (IVACG), XXII Meeting (2004) Vitamin A and the Common Agenda for Micronutrients. USAID. Rockville, Md., USA
Jayarajan P, Reddy V, Mohanram M (1980) Effect of dietary fat on absorption of beta carotene from green leafy vegetables in children. Indian J Med Res 71:53-56
Jayathunge K G, Stratakos A C, Cregenzan-Albertia O, Grant I R, Lyng J, Koidis A. Enhancing the lycopene in vitro bioaccessability of tomato juice synergistically applying thermal and non-thermal processing technologies. Food Chemistry 221 (2017) 698-705.
Jialal I, Devoraj S: Low-density lipoprotein oxidation, antioxidants, and atherosclerosis: A clinical biochemistry perspective. Clin Chem 1996; 42: 498-506.
Johnson, E. J., 2014. Role of lutein and zeaxanthin in visual and cognitive function throughout the lifespan. Nutr. Rev. 72 (9), 605-612.
Johnson E I, Qin J, Krinsky N I, and Russell, R M. 1997. Ingestion by men of a combined dose of beta-carotene and lycopene does not affect the absorption of beta-carotene but improves that of lycopene. J. Nutr., 127: 1833-1837.
Kim J Y, Paik J K, Kim O Y, Park H W, Lee J H, Jang Y, Lee J H. Effects of lycopene supplementation on oxidative stress and markers of endothelial function in healthy men. Atherosclerosis. 2011 March; 215(1):189-95.
Kim, Y.; Park, Y.; Lee, K.; Jeon, S.; Gregor, R. & Choi, S. (2012): Dose dependent effects of lycopene enriched tomato wino on liver and adipose tissue in high fat diet fed rats. Food Chemistry, 130: 42-48.
Kim Y, Takahashi H, Goto T et al. 9-oxo-10(E)-octadecadienoic acid derived from tomato is a potent PPARα agonist to decrease triglyceride accumulation in mouse primary hepatocytes. Molecular Nutrition & Food Research, 2010; DOI:10.1002/mnfr.201000264.2010.
Knockaert G, Pulissery S, Colle I, Van Buggenhout S, Hendrickx M, Loey A. 2012. Lycopene degradation, isomerization and in vitro bioaccessibility in high pressure homogenized tomato puree containing oil: Effect of additional thermal and high-pressure processing. Food Chemistry 135 (2012) 1290-1297
Krinsky N, Johnson E. 2005. Carotenoid actions and their relation to health and disease. Mol. Asp. Med. 26, 459-516
Kritchevsky S B. (1999). B-Carotene, carotenoids and the prevention of coronary heart diseases. J. Nutr. 129:5-8.
Kuczora, S. Richardson, P. (2012) “Industry position papers—Fortification” Head of Department Food Manufacturing Technologies. Available at: http://www.foodhealthinnovation.com/media/6508/industry_position_paper_-_fortification.pdf. Accessed on July 2019
Kumnar P, Coronel P, Simunovic J, Sandeep K P. Thermophysical and Dielectric Properties of Salsa con queso and its Vegetable Ingredients at Sterilizaiton Temperatures. 2008. International Journal of Food Properties, 11: 112-126.
Landrum J T, Bone R A. (2001). Lutein, zeaxanthin and the macular pigment. Arch. Biochem. Biophys. 385:28-40.
Lavy A, Ben Amotz A, and Aviram M. 1993. Preferential inhibition of LDL oxidation by the all-trans isomer of beta-carotene in comparison with 9-cis beta-carotene. Eur. J. Clin. Chem. Clin. Biochem., 31: 83-90.
Lee A, Thurnham D I, Chopra M (2000) Free Radical Biol Med 29:1051-1055
Lieblein-boff, J. C., et al., 2015. Exploratory metabolomic analyses reveal compounds correlated with lutein concentration in frontal cortex, hippocampus, and occipital cortex of human infant brain. PLoS One 1-19.
Lopez A. 1987. A complete course in canning and related processes. Book III. Processing procedure for canned products. Baltimore, Md. The Canning Trade. p. 96
Lu, Y, Etoh, H, and Watanaba, N. 1995. A new carotenoid, hydrogen peroxide oxidation product from lycopene. Biosci. Biotechnol. Biochem., 59:2153-2155.
Mackley M R, Marsall R T J, Smeulders J B A F. Zhao F D. (1994). The rheological characterization of polymeric and colloidal fluids. Chemical Engineering Science 49: 2551-2565.
Marini, A., & Gragnolati, M. (2003). Malnutrition and poverty in Guatemala. World Bank. http://siteresources.worldbank.org/EXTLACREGTOPNUT/Resources/Guatemala_nurtition_WPS2967.pdf.Accessed July, 2019
Marsh G L, Leonard S J, Buhlert J E. (1979). Yield and quality of catsup produced to a standard solids and consistency level II. Influence of handling practices, break temperature and cultivar. Journal of Food Processing Preservation 3: 195-212.
Miller, l N J, Sampson, J, Cadeias, J P, Bramley, P M, and Rice-Evans, C A. 1996. Antioxidant activities of carotenes and xanthophylls., FEBS.-Lett., 384:240-242
Mortenson, A, Skibsted, L H, Sampson, J, Rice-Evan, C, and Everett, S A. 1997. Comparative mechanisms and rates of free radical scavenging by carotenoid antioxidants. FEBS Lett., 418:91-97.
NC Food Processing and Manufacturing Initiative. NC State University and NC Dept. of Agriculture & Consumer Affairs (2015). Accessed on July 2019 https://harvest.cals.ncsu.edukkfinder/userfiles/files/The%20North%20Carolina%20Food%20Pro cessing%20and%20Manufacturing%20Initiative%20-%20An%20Economic%20Feasibilty%20Study%20012615.pdf
North Carolina Sweetpotato Commission. (2016). Industry. Retrieved from http://www.ncsweetpotatoes.com/sweet-potato-industry/. Accessed on July, 2019
Nguyen M, Francis D, Schwartz S. 2001. Thermal isomerization susceptibility of carotenoids in different tomato varities. J Sci Food Agric 81:910-917
Nguyen M L, Schwartz S J: Lycopene: chemical and biological properties. Food Technol 1999; 53:38-44.
Nguyen M, Francis D, Schwartz S. 2001. Thermal isomerization susceptibility of carotenoids in different tomato varieties. J Sci Food Agric 81: 910-7.
Omoni, O. & Aluko, R. (2005): The anticarcinogenic and antiatherogenic effects of lycopene: a review. Trends in Food Science & Technology, 16: 344-350.
Ozawa, Y., et al., 2012. Neuroprotective effects of lutein in the retina. Curr. Pharm. Des. 18, 51-56 Paganga G, Miller N, Rice-Evans C (1999) Free Radical Res 30:153-162
Page D, Van Stratum E, Degrou A, Renard CMGC (2012) Kinetics of temperature increase during tomato processing modulate the bioaccessibility of lycopene. Food Chemistry 135 (2012) 2462-2469
Parthasarathy S: Mechanisms by which dietary antioxidants may prevent cardiovascular disease. J Med Food 1998; 1:45-51.
Rao A V, Waseem Z, Agarwal S: Lycopene contents of tomatoes and tomato products and their contribution to dietary lycopene. Food Res Intl 1998; 31:737-741
Rao A V, Shen H: Effect of low dose lycopene intake on lycopene bioavaialbility and oxidative stress. Nutr Res 2002; 22: 1125-1131.
Rao M A, (1977). Measurement of flow properties of fluid foods-developments, limitations and interpretation of phenomena. Journal of Texture Studies 8:257-282
Ramaswamy, H S. Ohmic heating in food processing. Boca Raton, Fla.: CRC Press 2014. ISBN 9781420071092. OCLC 872623115
Rasmussen, W. D., 1968. Advances in American agriculture: The mechanical tomato harvester as a case study. Technology and Culture 9, 531-543
Reboul E and P. Borel. 2011 Proteins involved in uptake, intracellular transport and basolateral secretion of fat-soluble vitamins and carotenoids by mammalian enterocytes, Prog. Lipid Res., 50, 388-402
Reboul, E.; Richelle, M.; Perrot, E.; Desmoulins-Malezet, C.; Pirisi, V.; Borel, P. Bioaccessibility of carotenoids and vitamin E from their main dietary sources. J. Agric. Food Chem. 2006, 54, 8749-8755
Renzi, L. M., et al., 2014. Neurobiology of aging relationships between macular pigment optical density and cognitive function in unimpaired and mildly cognitively impaired older adults. Neurobiol. Aging 35 (7), 1695-1699.
Ribaya-Mercado I D, Ordovas, I M, and Russell R M. 1995. Effect of n-carotene supplementation on the concentrations and distributions of carotenoids, vitamin E, and vitamin A, and cholesterol in plasma lipoprotein and nonlipoprotein fractions in healthy older women. J. Am. Coll. Nutr., 14:614-620.
Richardson, P. (2001) Thermal technologies in food processing. Boca Raton, Fla.: CRC Press Inc. (pp 163-285)
Rock C L, Lovalvo J L, Emenhiser C, Ruffin M T, Flatt S W, Schwartz S J, 1998. Bioavailability of β-carotene is lower in raw than in processed carrots and spinach in women. J. Nutr. 128:913-916
Rohde L E, Hennekens C H, Ridker P M. Survey of C-reactive protein and cardiovascular risk factors in apparently healthy men. Am J Cardiol 1999; 84:1018-22.
Roger V L, Go A S, Lloyd-Jones D M, et al. Heart disease and stroke statistics-2012 update: a report from the American Heart Association. Circulation 2012; 125:e2-220.
Sanchez M C, Valencia C, Ciruelos A, Latorre A, Gallegos C. (2002). Influence of processing on the rheological properties of tomato paste. Journal of Science and Food Agriculure 82: 990-997
Santanam N, Song M, Rong R, Murphy A A, and Parthasarathy S. 2002. Atherosclerosis, oxidation, and endometriosis. Free Radic. Res., 36: 1315-1321
Schachter D, Finkelstein J D, Kowarski S. 1964, Metabolism of Vitamin D. I. Preparation of Radioactive Vitamin D and Its Intestinal Absorption in the Rat, J. Clin. Invest., 43, 787-796
Schierle J. Bretzel W, Biihler I, Faccin N, Hess D, Steiner H, Schuep W: Content and isomeric ratio of lycopene in food and human blood plasma. Food Chem 1996; 3:459-465.
Schmitz H H, Poor C L, Gugger E T, Erdman J W Jr. 1993. Analysis of carotenoids in human and animal tissues, Methods Enzymol., 214:102-116.
Schultheis, J. R. (2018). State Report—North Carolina. National Sweetpotato collaborators group, progress report. Presented on Jan. 20, 2018, Hilton Wilmington Riverside Hotel, Wilmington, N.C.
Schwedhelm E., Maas R., Troos R., and Boger R. 2003. Clinical Pharma-cokinetics of antioxidants and their impact of systemic oxidative stress. Pharmacokinet, 42:437-459.
Shi J, Maguer M L: Lycopene in tomatoes: chemical and physical properties affected by food processing. Food Sci Nutr 2000; 40: 1-42
Shi J, Maguer M L. Bryan M. Kakudo Y: Kinetics of lycopene degradation in tomato puree by heat and light irradiation. J Food Process Eng 2003; 25:485-498.
Shi J, Wu Y, Bryan M, Maguer M L: Oxidation and isomerization of lycopene under thermal treatment and light irradiation in food processing. Nutraceut Food 2002; 7: 179-183.
Stahl W, Sies H: Uptake of lycopene and its geometric isomers is greater from heat-processed than from unprocessed tomato juice in humans. J Nutr 1992; 122:2161-2166.
Stewart A J, Bozonnet S, Mullen W, Jenkins G I, Lean M E, Crozier A (2000) J Agric Food Chem 48:2663-2669
Tang J. (2005). Dielectric Properties of Foods. In: Schubert H and Regier M (eds) Microwave Processing of Foods Cambridge: Woodhead Publishing, pp. 22-38.
Truong V D. 1992. Sweetpotato beverages: product development and technology transfer. In: Hill W A, Bonsi C K, Loretan P A, editors. Sweetpotato technology for the 21st century. Proc Intern Symp; 1991 Jun. 2-6. Tuskegee, Ala.: Tuskegee Univ. p 389-399.
Truong, V.-D., & Avula, R. Y. (2010). Sweetpotato purees and powders for functional food ingredients. In Ray, R. C. & Tomlins, K. I. (Eds.), Sweetpotato: Post Harvest Aspects in Food (117-161). New York, N.Y.: Nova Science Publishers, Inc.
Truong V D, Walter W M, Giesbrecht F G. 1995. Texturization of sweetpotato puree with alginate/calcium: optimization by response surface methodology. J Food Sci 60:1054-9, 1074.
Tsimikas S, Bergmark C, Beyer R W, Patel R, Pattison J, Miller E, Juliano J, and Witztum J L. 2003. Temporal increases in plasma markers of oxidized low-density lipoprotein strongly reflect the presence of acute coronary syndromes. J. Am. Coll. Cardiol., 41:360-370.
Tulchinsky, T. H. (2010). Micronutrient deficiency conditions: Global health issues. Public Health Reviews, 32(1), 243-255
Tyssandier V, Cardinavlt N, Caris-Veyrat C, Amiot M I, Grolier P, Bouteloup C, Azais-Braesco V, and Borel P. 2002. Vegetable-borne lutein, lycopene, and beta-carotene compete for incorporation into chylomicrons, with no adverse effect on the medium-term (3 wk) plasma status of carotenoids in humans. Am. J Clin. Nutr., 75:526-534.
Tyssandier, V.; Reboul, E.; Dumas, J.-F.; Bouteloup-Demange, C.; Armand, M.; Marcand, J.; Sallas, M.; Borel, P. Processing of vegetable-borne carotenoids in the human stomach and duodenum. Am. J. Physiol. Gastrointest. Liver Physiol. 2003, 284, 913-923
Unlu N Z, Bohn T, Francis D M, Nagaraja H N, Clinton S K, Schwartz S J. Lycopene from heat-induced cis-isomer-rich tomato sauce is more bioavailable than from all-trans-rich tomato sauce in human subjects. British Journal of Nutrition (2007), 98, 140-146.
United States Department of Agriculture. (2017). Food Security in the U.S. Retrieved from https://www.ers.usda.gov/topics/food-nutrition-assistance/food-security-in-theus/definitions-of-food-security/. Accessed on July, 2019
United States Department of Agriculture, (2017) Census of Agriculture—State Data North Carolina—National Agriculture Statistics Service. https://www.nass.usda.gov/Publications/AgCensus/2017/Full_Report/Volume_1,_Chapter_1_Sta te_Level/North_Carolina/st37_1_0036_0036.pdf. Accessed on July 2019
USDA/NCC Carotenoid Database for U.S. Foods (1998 & Tomato Research Council
Valdes, D. N., 1994. Machine Politics in California Agriculture, 1945-1990s. Pacific Historical Review 63, 203-224
Van het Hof K, de Boer B, Tijburg L, Lucius B, Zijp I, West C, Hautvast J, Weststrate J. 1999. Carotenoid bioavailability in humans from tomatoes processed in different ways determined from the carotenoid response in the triglyceride-rich lipoprotein fraction of plasma after a single consumption and in plasma after four days of consumption. American Society of Nutritional Sciences 1189-1196
Van Zeben W, Hendriks T F, 1948 The absorption of carotene from cooked carrots. Zschr. Vitamin Forsch. 19: 265-266
Velmurugan B, Bhuvaneswari V, and Nagini S. 2002. Antiperoxidative effects of lycopene during N-methyl-N′-nitro-N-nitrosoguanidine-induced gastric carcinogenesis. Fitoterapia., 73:604-611.
Venkatesh M S, Raghavan G S V, (2004) An overview of microwave processing and dielectric properties of agri-food materials. Biosystems Engineering 88(1): 1-18
Victoria, N. G., O. M. C. van der Valk, and A. Elings. 2011. Mexican Protected Horticulture: Production and Market of Mexican Protected Horticulture Described and Analysed (No. 1126). Wageningen U R Greenhouse Horticulture/LEI, Wageningen, The Netherlands.
Vinson J A, Hao Y, Su X Zubik L (1998) J Agric Food Chem 46:3630-3634
Vishwanathan, R., Johnson, E. J., 2013. Lutein and zeaxanthin and eye disease. In: Tanumihardjo, S. A. (Ed.), Caroltenoids and Human Health. Springer Science and Business Media, New York, pp. 215-235.
Vishwanathan, R., Iannaccone, A., et al., 2014b. Macular pigment optical density is related to cognitive function in older people. Age Ageing 43, 271-275.
Vishwanathan, R., Kuchan, M. J., et al., 2014a. Lutein and preterm infants with decreased concentrations of brain carotenoids. Hepatol. Nutr. 59 (5), 659-665.
Visser M, Bouter L M, McQuillan G M, Wener M H, Harris T B. Elevated C-reactive protein levels in overweight and obese adults. JAMA 1999; 282:2131-5.
Walter W M, Truong V D, Sylvia K E. 2001. Methods for producing cooked sweetpotato products and compositions thereof. Washington, D.C.: USDA. U.S. Pat. No. 6,197,363 B1. Issued Mar. 6, 2001.
Wang, S., Nie, S., & Zhu, F. (2016). Chemical constituents and health effects of sweetpotato. Food Research International, 89, 90-116
Wang X D. Carotenoids. In: Ross C A, Caballero B, Cousins R J, Tucker K L, Ziegler T R, eds. Modern Nutrition in Health and Disease. 11^{th} ed: Lippincott Williams & Wilkins; 2014:427-439
Wang Y, Cui R, Xiao Y, Fang J, Xu Q (2015) Effect of Carotene and Lycopene on the Risk of Prostate Cancer: A Systematic Review and Dose-Response Meta-Analysis of Observational Studies. PLoS ONE 10(9): e0137427. Doi:10.1371/journal.pone.0137427
Wegner C, John M. 2006. Thermal isomerization of lycopene. Ludwigshafen, D E.: BASF Aktiengesellschaft U.S. Pat. No. 7,126,036. Issued Oct. 24, 2006
Weisburger J H: Lycopene and tomato products in health promotion. Exp Biol Med. 2002; 227:924-927.
White W S, Peck K M, Bierer T L, Gugger E T, and Erdman J W Jr. 1993. Interactions of oral beta-carotene and canthaxanthin in ferrets. J. Nutr., 123: 1405-1413.
Woolfe J A. 1992. Sweetpotato: an untapped food resource. Cambridge, U.K.: Cambridge Univ. Press.
World Health Organization and Food and Agriculture Organization of the United Nations. (2006). Guidelines on food fortification with micronutrients. Available at http://www.who.int/nutrition/publications/guide_food_fortification_micronutrients.pdf. Accessed on July 2019
Wu, F., Z. Guan, and M. Garcia-Nazariega. 2017. Comparison of Labor Costs between Florida and Mexican Strawberry Industries. FE1023. Gainesville, Fla.: University of Florida, Institute of Food and Agricultural Sciences. http://edis.ifas. ufl.edu/fe1023.
Yeh S, and Hu M. 2000. Antioxidant and pro-oxidant effects of lycopene in comparison with β-carotene on oxidant-induced damage in Hs68 cells. J. Nutr. Biochem., 11:548-554.
Yanggen, D., & Nagujja, S. (2006). The use of orange-fleshed sweetpotato to combat Vitamin A deficiency in Uganda. A study of varietal preferences, extension strategies and postharvest utilization. International Potato Center (CIP). Lima, Peru: CIP Communication and Public Awareness Department (CPAD). Retrieved from http://cipotato.org/cipotato/publications/pdf/003247.pdf
Zhu, M., Z. Guan, and F. Wu. 2013. “An Overview of the US Tomato Industry.” 2013 Tomato Institute Proceedings

### DESCRIPTION OF THE FIGURE

FIG. 1 illustrates the structural distinctions of the predominant lycopene geometrical isomers. Geometric isomers of lycopene, all-trans lycopene, 15-cis lycopene, 13-cis lycopene, 11-cis lycopene, 9-cis lycopene, 7-cis lycopene, 5-cis lycopene

FIG. 2 illustrates the quantification of 7-cis-lycopene detected in processed Roma Tomatoes for unheated control, heated, heated with oil and heated with oil and lecithin. The data shows a 344% increase of 7-cis-Lycopene in HR (heated roma) compared to control (unheated) roma.

FIG. 3 illustrates the quantification of 9-cis-lycopene detected in processed Roma Tomatoes for unheated control, heated, heated with oil and heated with oil and lecithin. The data shows a 1010% increase of 9-cis-lycopene in HR (heated roma) compared to control (unheated). 9-cis lycopene in HRO (heated roma w/oil) increased by 758% compared to control. 9-cis lycopene in HROL increased by 333% compared to untreated control.

FIG. 4 illustrates the quantification of 9′-cis-lycopene detected in processed Roma Tomatoes for unheated control, heated, heated with oil and heated with oil and lecithin. The data shows a 50% increase of 9′-cis-lycopene in HRO (Heated Roma Oil) compared to control (unheated).

FIG. 5 illustrates the quantification of 13-cis-lycopene detected in processed Roma Tomatoes for unheated control, heated, heated with oil and heated with oil and lecithin. The data shows a 40000% increase of 13-cis-lycopene in HRO (Heated Roma Oil) compared to control (unheated)

FIG. 6 illustrates the quantification of di-cis-lycopene detected in processed Roma Tomatoes for unheated control, heated, heated with oil and heated with oil and lecithin. The data shows a 99.4% increase of di-cis-Lycopene isomer in HR (heated roma) compared to control (unheated). Data shows a 182% increase of di-cis-Lycopene isomer in HRO compared to control. Data shows an 81.9% increase of di-cis-Lycopene isomer in HROL compared to control

FIG. 7 illustrates the quantification of c-lycopene-1 detected in processed Roma Tomatoes for unheated control, heated, heated with oil and heated with oil and lecithin. Data shows a 350% increase of c-Lycopene-1 in HRO compared to control; Data displays a 270% increase of c-Lycopene-1 in HROL (Heated Roma Oil/Lecithin) compared to control.

FIG. 8 illustrates the quantification of 5-cis-lycopene detected in processed Roma Tomatoes for unheated control, heated, heated with oil and heated with oil and lecithin. Stable form 5-cis-lycopene decreased in HR, HRO and HROL by 8.7%, 29.5%, 21.5% (respectively) compared to control.

FIG. 9 illustrates the quantification of trans-lycopene detected in processed Roma Tomatoes for unheated control, heated, heated with oil and heated with oil and lecithin. Stable form trans-lycopene remained constant in HR compared to control. HRO and HRL expressed 31.9% and 9.64%, respectively, decreased trans-Lycopene content compared to control.

FIG. 10 illustrates the quantification of cis-Lycopene Absolute Bioaccessibility identified in processed Roma Tomatoes for unheated control, heated, heated with oil, heated with oil/lecithin. Data shows absolute bioaccessibility of total cis-Lycopene increase 200% in HRO compared to control; Data displays a 650% increase in total cis-Lycopene absolute bioaccessibility in HROL (Heated Roma Oil/Lecithin) compared to control.

FIG. 11 illustrates the quantification of trans-Lycopene Absolute Bioaccessibility identified in processed Roma Tomatoes for unheated control, heated, heated w/oil, heated w/oil+lecithin. Data shows absolute bioaccessbility of trans-Lycopene decreased 20% in HR compared to control, Trans-Lycopene absolute bioaccessibility increased 220% in HRO compared to control, and increased 850% in HROL compared to control

FIG. 12 illustrates the quantification of % Relative Bioaccessibility (aqueous/digestia) calculated in processed Roma Tomatoes for unheated control, heated, heated w/oil, heated w/oil+lecithin. Relative bioaccessbility of cis-lycopene increased by 25% in HR compared to control. Cis-Lycopene relative bioaccessbility increased in HRO by 100% compared to control. Cis-Lycopene in HROL increased by 362.8% compared to control. Total-Lycopene relative bioaccessbility remained constant in HR, compared to control but increased by 315.5% in HRO compared to control, increased by 905.6% in HROL compared to control

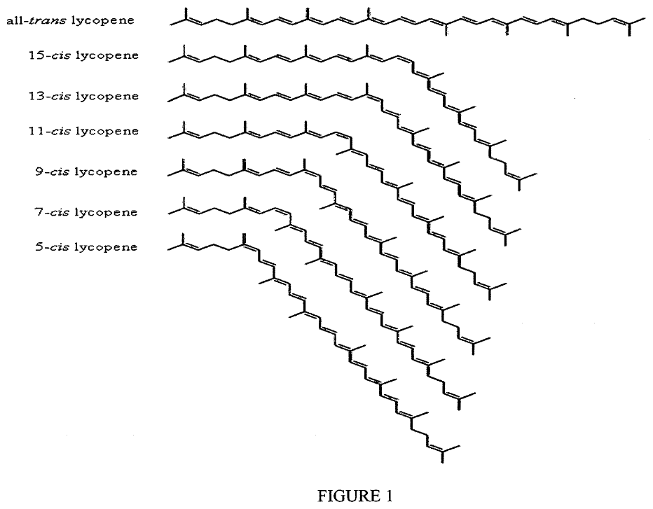

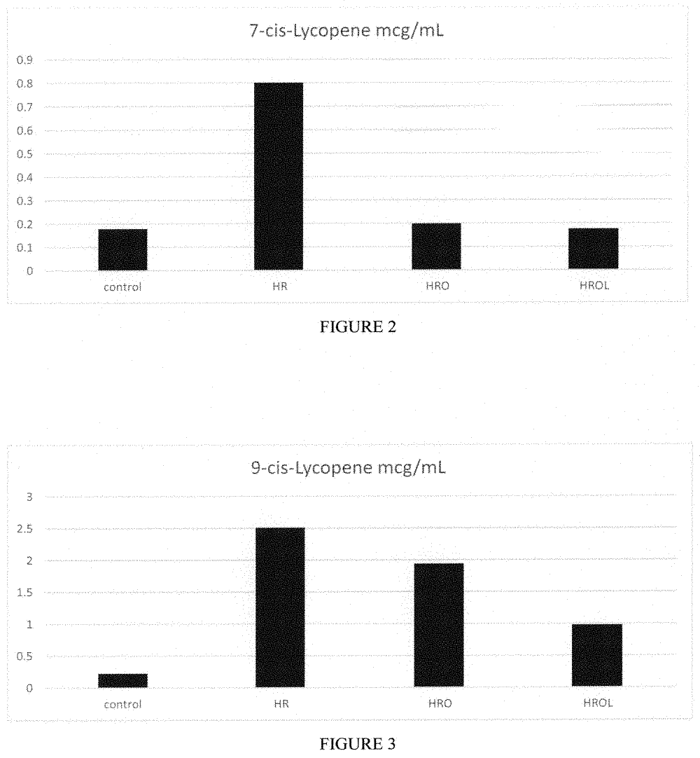

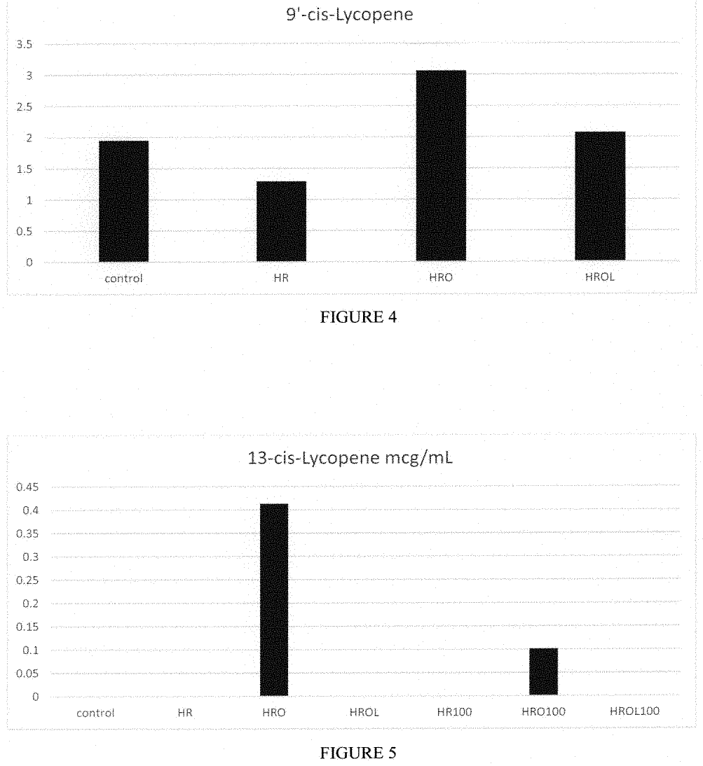

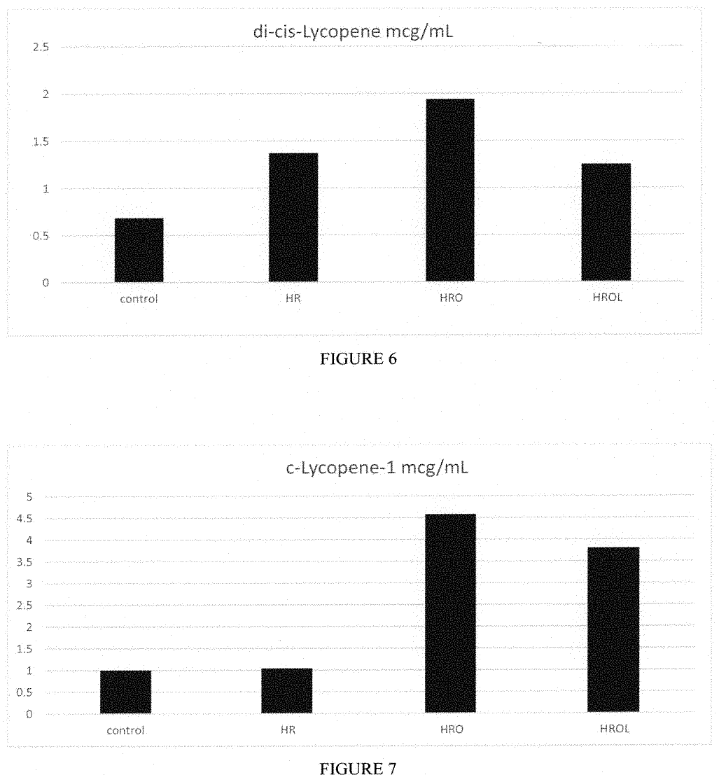

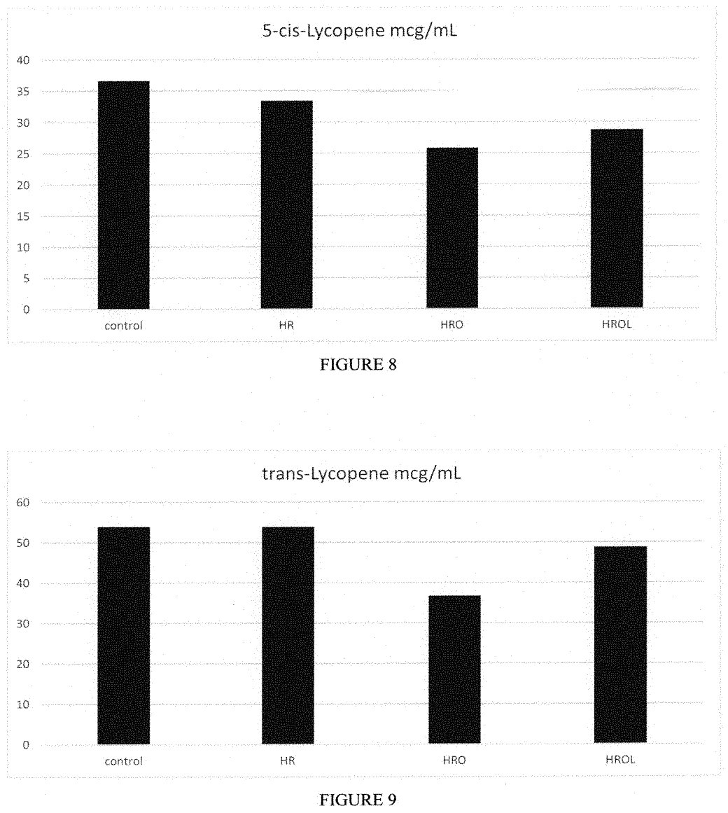

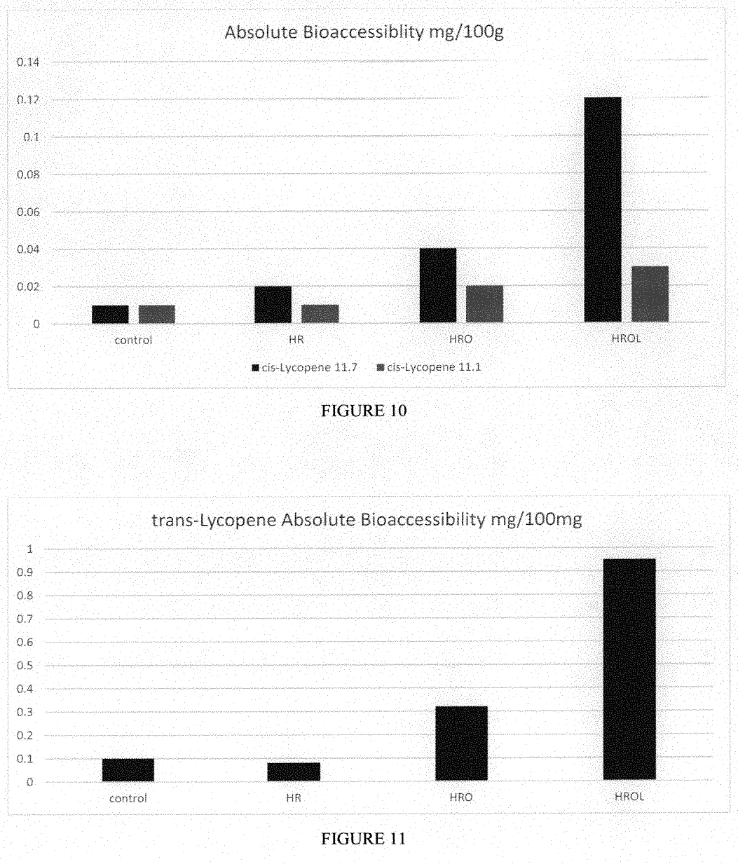

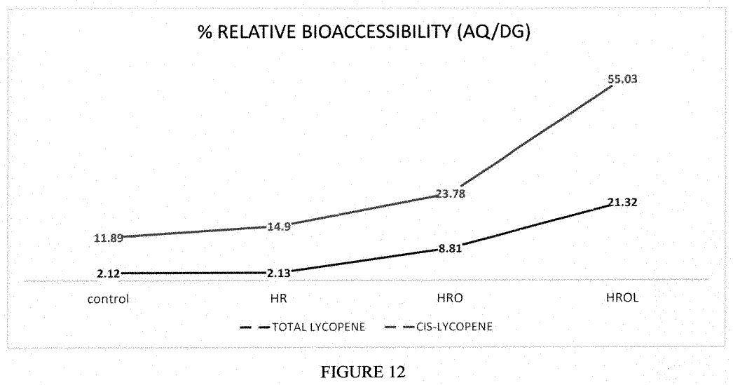

We claim:

1. A process for the rapid thermal isomerization of a mixture of all-trans-lycopene and its cis-isomers of any composition to increase the proportion of cis-isomers, wherein the isomerization takes place in a polar solvent.

2. A functional ingredient, nutraceutical, functional food, medical food composition suitable for administration to mammal, comprising greater than 4% of isomerized cis-lycopene compared to total lycopene content.

3. A process claimed in claim 1, wherein the isomerization takes place by way of energizing polar water molecules and ionic salt compounds through friction within the food mixture matrix using microwave-assisted heating

4. A process claimed in claim 1, wherein the isomerization takes place by way of energizing food mixture matrix using volumetric heating.

5. A process as claimed in claim 1, wherein the isomerization takes place at between 60 and 180 degree C.

6. A process as claimed in claim 1, wherein the monounsaturated and polyunsaturated fatty acids such as edible oils including but not limited to canola oil, olive oil, sunflower oil, safflower oil, saffron oil, vegetable oil, grapeseed oil, black cumin seed oil, cooking oils, omega 3 fatty acids, omega 6 fatty acids, fat soluble vitamins E, D, K, A are used as solvent components and processing aids in water used as solvent.

7. A process as claimed in any of claim 1, wherein C.sub.1 C.sub.8-alcohols, diols, polyols, amides, carbonates, sulfoxides, or water are used as solvent.

8. A process as claimed in any of claim 1, wherein methanol, ethanol, isopropanol, butanol or organic solvents are used as solvent or processing aids.

9. A process as claimed in any of claim 1, wherein surfactants, including but not limited to proteins, amino acids, lecithin, monoglycerides, diglycerides, triglycerides, fat soluble vitamins E, D, K, A used as solvent or processing aids.

10. A process as claimed in any of claim 1, wherein cation other than H, preferably Na.sup+ or K.sup+ or Li.sup+ or Iodine is used as a processing aid to increase isomerization potential or enhance dielectric properties though out carotenoid solvent matrix.

11. A process as claimed in any of claim 1, wherein less than 97% of total lycopene is in the all-trans-lycopene crystalline form at the chosen isomerization temperature.

12. A process as claimed in any of claim 1, wherein more than 4% of the total lycopene content is in its cis-lycopene isomeric form at the chosen isomerization temperature.

13. A process as claimed in any of claim 1, where in 0.2% or more of trans-Lycopene is converted to cis-lycopene isomeric form at the chosen isomerization temperature.

13. A process as claimed in claim 1, wherein the lycopene mixture comprises 5-cis lycopene.

14. A process as claimed in claim 1, wherein the lycopene mixture may comprise 7-cis lycopene

15. A process as claimed in claim 1, wherein the lycopene mixture may comprise 9-cis lycopene.

16. A process as claimed in claim 1, wherein the lycopene mixture may comprise 11-cis lycopene.

17. A process as claimed in claim 1, wherein the lycopene mixture may comprise 13-cis lycopene.

18. A process as claimed in claim 1, wherein the lycopene mixture may comprise 15-cis lycopene.

19. A process as claimed in claim 1, where said carrier is a buffered solution having a pH greater than 2.0
